(19) United States
(12) Patent Application Publication
(10) Pub. No.: US 2026/0018968 A1
(43) Pub. Date: Jan. 15, 2026

(54) SYSTEMS AND METHODS FOR SMART CARBON CAPTURE AND POWER GENERATION

(71) Applicant: Purdue Research Foundation, West Lafayette, IN (US)

(72) Inventors: Helber Antonio Esquivel-Puentes, Lafayette, IN (US); Luciano Castillo, Carmel, IN (US); Maria Mirian Velay-Lizancos, West Lafayette, IN (US); Carsten Hein Westergaard, Houston, TX (US); Jhon Quinones, Den Haag (NL)

(73) Assignee: Purdue Research Foundation, West Lafayette, IN (US)

(21) Appl. No.: 19/256,858

(22) Filed: Jul. 1, 2025

Related U.S. Application Data
(60) Provisional application No. 63/670,483, filed on Jul. 12, 2024.

(51) Int. Cl. H02K 7/18; G01N 33/00; G01P 5/24; G01P 13/04

(52) U.S. Cl. CPC H02K 7/183; G01N 33/0027; G01P 5/24; G01P 13/045

(57) ABSTRACT

A carbon capture and power generation system includes a manifold, a pair of air foil blades, an electric generation system, a yaw system, one or more environmental sensors, and a processor. The pair of air foil blades are spaced apart to allow environmental air flow between them, and each air foil blade includes a plurality of orifices on the exterior surface. The electric generation system outputs and electric power signal as its propellor rotates, and the yaw system rotates the air foil blades. The environmental sensors monitor environmental conditions and generate respective output data signals. The processor accesses a neural network trained to receive the output data signals from the one or more environmental sensors and generate rotational control signals to the yaw system to rotate the air foil blades based upon the output data signals, and the processor transmits the rotational control signals to the yaw system.

### CROSS-REFERENCE TO RELATED APPLICATIONS

This application is related to and claims the priority benefit of U.S. Provisional Application No. 63/670,483, entitled “Systems and Methods For Smart Carbon Capture and Power Generation” filed Jul. 12, 2024, the contents of which are hereby incorporated by reference in their entirety into the present disclosure.

### TECHNICAL FIELD

The present application relates to carbon capture and power generation, and specifically to systems which dynamically reposition based on environmental conditions for generating electric power from and capturing CO₂ from polluted air that moves between passive airfoils.

### BACKGROUND

In recent years, the growth of urbanization has brought unprecedented challenges to the environmental landscape, none more pressing than the escalating levels of carbon dioxide (CO₂) emissions within urban environments such as cities. As populations surge and industries expand, urban centers have become epicenters of CO₂ production, contributing significantly to global warming and climate change. This surge in CO₂ emissions not only threatens the delicate balance of our planet's ecosystem but also poses grave risks to the health and well-being of city dwellers. The CO₂ problem in urban areas requires innovative solutions to mitigate its harmful effects while the energy source transition to renewables is advanced.

### SUMMARY

Described herein is a technical solution for smart carbon capture and electric power generation.

In one aspect of the described embodiments, a system is provided, which can include a pair of air foil blades, an electric generation system, a manifold, a yaw system, one or more environmental sensors, and a processing system. The pair of air foil blades can be spaced apart to allow environmental air flow between them, and each air foil blade can include a hollow interior cavity and a plurality of orifices on an exterior surface of the air foil blade. Each orifice can be configured to enable air flow from an exterior position relative to the air foil blade through the orifices and into the hollow interior cavity. The electric generation system can include at least a rotor and a propellor coupled with the rotor and can be configured to generate and output and electric power signal as the propellor rotates. The manifold can include a first fluid reservoir for containing an aqueous solution which can be adapted to capture CO₂ from the air flowing through the system. The yaw system can be selectively operable to rotate the air foil blades. The one or more environmental sensors can be configured to monitor one or more environmental conditions and generate respective output data signals. The processing system can be configured to access a neural network trained to receive the output data signals from the one or more environmental sensors and generate rotational control signals to the yaw system to rotate the air foil blades based upon the output data signals, and the processing system can selectively transmit the rotational control signals to the yaw system.

In another aspect of the described embodiments, a method is provided, which can comprise: receiving, by a processing system, environmental condition data from one or more environmental sensors; inputting, by the processing system, the environmental condition data into a neural network trained to generate control signals for orienting a pair of air foil blades based upon the environmental condition data, the pair of air foil blades being spaced apart, configured to enable environmental air flow between them; transmitting, by the processing system, the control signals to a yaw system to selectively rotate the pair of air foil blades so as to orient the air foil blades; generating electric power from a propellor coupled to a rotor when the propellor rotates as the air flows between the oriented air foil blades into a column housing the propellor and the rotor; and capturing CO₂ from the air flowing through a system of the air foil blades and the column, by directing the air flow through a manifold containing an aqueous solution adapted to capture CO₂.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an exemplary power generation and carbon capture system according to an example embodiment of the present disclosure;

FIG. 2 illustrates a block diagram of the power generation and carbon capture system of FIG. 1 according to an example embodiment of the present disclosure;

FIG. 3 depicts an example application of the power generation and carbon capture system of FIG. 1 showing multiple such systems placed on top of a building according to an example embodiment of the present disclosure;

FIG. 4 illustrates a flow chart of a method for power generation and carbon capture according to an example embodiment of the present disclosure; and

FIG. 5 depicts an example operational schematic showing the processor utilizing the neural network architecture for receiving data indicative of environmental conditions around the power generation and carbon capture system and generating control signals configured to adjust the positioning of the power generation and carbon capture system according to the environmental conditions.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown, or the precise experimental arrangements used to arrive at the various graphical results shown in the drawings.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following described teachings, expressions, embodiments, examples, etc., should, therefore, not be viewed in isolation relative to each other. Various suitable ways in which the teachings herein may be combined will be readily apparent to those of ordinary skill in the art in view of the teachings herein. Such modifications and variations are intended to be included within the scope of the claims.

The sources of CO₂ in the cities are often high population densities, extensive industrial activity, and heavy traffic congestion, all of which contribute to elevated CO₂ levels in their atmospheres. Large, densely populated cities such as Los Angeles, California can emit around 100 million metric tons of CO₂ per year. Further, cities like Los Angeles include many tall buildings which are strong source areas for harnessing wind energy. Efficient utilization of renewable (e.g., wind) energy combined with the reduction of CO₂ emissions are both important for mitigating climate change and promoting sustainable development. However, conventional wind turbine control systems often lack adaptability to rapidly changing environmental conditions, therefor limiting their effectiveness in maximizing energy production alongside their CO₂ filtration functions.

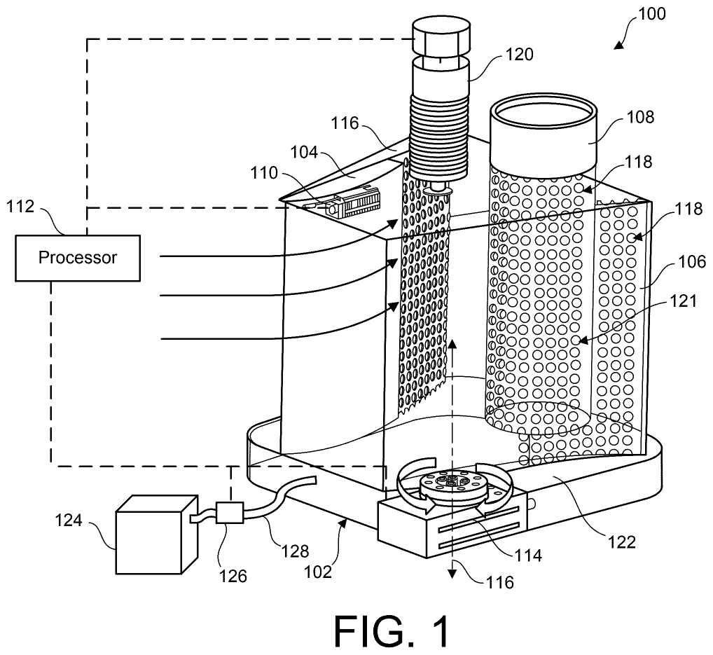

To that end, shown in FIG. 1 is a smart and efficient wind-harvesting and CO₂ capturing system 100. The system 100 includes a manifold 102, a mirrored pair of passive airfoils (air foil blades) 104, 106, and a column 108 which houses a wind-turbine electric generation system comprising at least a rotor and a propellor that is coupled to the rotor, and optionally additional components forming the wind-turbine electric generation system (e.g., a gearbox, generator, controller, brake, electrical wiring, etc.). Accordingly, the wind-turbine generator is sheltered from the weather. The column may be positioned between the air foil blades, as particularly shown in FIG. 1.

The manifold 102 includes a fluid reservoir 122 which may also contain an aqueous solution to capture carbon from the air flowing through the system 100. The aqueous solution may include calcium hydroxide to aid in the carbon capture from the air flowing through the system 100. The airfoils 104, 106 are formed with a hollow interior with a cap 116, and a plurality of orifices 118 on their inward facing surfaces for air to pass through. The column 108 also includes a plurality of orifices 121 on its exterior surface. Optionally, at least external surfaces of the airfoils, the cap 116, and/or the column 108 are at least partially covered with a titanium dioxide (TiO₂) coating to disintegrate NOx in the surrounding air.

Referring more specifically to the structural arrangement shown in FIG. 1, the system 100 demonstrates the integration of multiple subsystems working in concert to achieve both power generation and CO₂ capture functionality. The manifold 102 is positioned at the base of the system 100 and includes an inlet port (not shown) for receiving contaminated air and an outlet port (not shown) for discharging treated air. The manifold 102 further includes internal baffles (not shown) that direct the air flow through the aqueous solution to maximize contact time between the CO₂-laden air and the calcium hydroxide solution. The first airfoil 104 and second airfoil 106 are positioned symmetrically about the central column 108, each maintaining a predetermined spacing that creates the optimal low-pressure zone for air acceleration. In some embodiments, this spacing ranges from 0.5 to 2.0 meters, depending on the wind conditions and building height. In some embodiments, the spacing ranges from 1.0 to 1.5 meters for buildings between 50 and 100 meters in height. In some embodiments, the spacing is adjusted dynamically based on real-time wind measurements. The airfoils 104, 106 include internal support structures (not shown) that maintain their hollow integrity while enabling air flow through the orifices 118. The column 108 houses the complete wind-turbine electric generation system, including a nacelle assembly (not shown) that protects the internal components from environmental exposure while enabling air flow through the orifices 121. The configuration enables both enhanced wind energy capture through increased air flow velocity and simultaneous CO₂ treatment of the same air mass, achieving dual environmental benefits from a single installation.

Additionally, the system 100 can include at least one CO₂ sensor 110 and at least one environmental sensor 120, each of which communicably coupled with a processing system (e.g., a processor 112 which may be positioned locally and hard wired with the sensors 110, 120 or positioned remotely and configured to communicate with the sensors 110, 120 wirelessly). In some embodiments, the CO₂ sensor 110 may share collected CO₂ data to the processor 112 for data manipulation and user-viewing, or for control of the aqueous solution delivery to the manifold 102. Particularly, a second fluid reservoir 124 may be placed nearby and acting as the main aqueous solution reservoir and can include a pump 126 and tubing 128 to selectively transfer the aqueous solution to the manifold 102 as needed according to the collected CO₂ data. The pump 126 may be operated by the processor 112 upon the processor 112 analyzing the received CO₂ data.

The environmental sensor 120 (e.g., a sonic anemometer configured to monitor wind speed and direction) may be positioned anywhere on the system 100 or near the system 100, and a plurality of such sensors 120 may be strategically placed around the one or more systems 100 to monitor one or more environmental conditions. The system 100 further includes a yaw system 114 communicably coupled with the processing system, for example the processor 112, for selectively rotating the system 100 about an axis 116 to maximize the functionality of the wind generation according to the wind direction.

The environmental sensors 120 comprise industrial-grade measurement devices specifically selected for integration with the artificial intelligence algorithms and outdoor environmental durability requirements. In some embodiments, the sonic anemometer includes ultrasonic transducers positioned in three-dimensional arrays to measure wind speed and direction vectors with high temporal resolution suitable for the deep learning data processing. The transducers operate at frequencies between 40 and 200 kHz to provide measurements across varying atmospheric conditions without mechanical components that could introduce measurement noise or require frequent maintenance. In some embodiments, the sonic anemometer includes heating elements to prevent ice formation during winter conditions that could compromise measurement accuracy.

The sonic anemometer provides wind speed measurements in the range of 0 to 60 m/s with 0.01 m/s resolution and wind direction measurements with 0.1 degree resolution, meeting the accuracy requirements for effective neural network training and inference operations. In some embodiments, the anemometer includes internal calibration references and automatic drift compensation to maintain long-term measurement stability. In some embodiments, the anemometer incorporates temperature and humidity compensation algorithms to account for atmospheric effects on ultrasonic propagation characteristics.

In some embodiments, the CO₂ sensor 110 employs non-dispersive infrared (NDIR) sensing optimized for the concentration ranges and response times required by AI control algorithms. The sensor provides measurements in the range of 400 to 5000 ppm with 1 ppm resolution and response times under 30 seconds, enabling real-time feedback for the chemical process control systems. In some embodiments, the NDIR sensor utilizes dual-beam configurations to compensate for optical source aging and environmental drift effects. In some embodiments, the sensor includes automatic zero-point calibration using filtered ambient air references to maintain long-term accuracy without manual intervention.

In some embodiments, the yaw system 114 comprises servomotor assemblies with gear reduction mechanisms configured to provide precise positioning control under varying wind loads while maintaining compatibility with AI control system response requirements. In some embodiments, the gear reduction ratios range from 100:1 to 500:1 depending on the system size and the torque requirements for different installation configurations. In some embodiments, the servomotors include absolute position encoders that provide real-time orientation feedback with resolution better than 0.1 degrees, enabling precise control loop closure for the AI algorithms. In some embodiments, the yaw system 114 includes electromagnetic brakes that engage automatically during maintenance operations or extreme weather conditions.

The manifold 102 can comprise corrosion-resistant materials and internal configurations optimized for the calcium hydroxide chemical reactions described in the disclosure. In some embodiments, the manifold comprises stainless steel construction for resistance to the alkaline chemical environment. In some embodiments, the manifold includes polymer constructions such as high-density polyethylene (HDPE) or polypropylene for reduced weight and cost while maintaining chemical compatibility. The internal configuration includes flow distribution systems that ensure uniform contact between the air stream and the aqueous solution to maximize the chemical reaction efficiency.

The pump 126 includes variable flow rate control systems that interface with the AI processing algorithms to optimize chemical delivery based on real-time environmental conditions and CO₂ concentration measurements. In some embodiments, the pump 126 comprises peristaltic configurations that minimize chemical contact with mechanical components while providing precise flow control. In some embodiments, the pump 126 includes centrifugal configurations with chemical-resistant materials for higher flow rate applications. The flow control systems provide rates ranging from 0.1 to 10 liters per minute with accuracy within ±2% of the setpoint values, enabling precise stoichiometric control of the chemical reactions.

The airfoils 104, 106 comprise lightweight structural materials configured to withstand outdoor environmental conditions while maintaining the acrodynamic properties required for the enhanced wind energy capture described in the disclosure. In some embodiments, the airfoils 104, 106 comprise aluminum alloy constructions with internal reinforcement structures that provide structural integrity while minimizing weight. In some embodiments, the airfoils 104, 106 include composite material constructions such as carbon fiber or fiberglass-reinforced plastics that offer enhanced strength-to-weight ratios and resistance to environmental degradation. The aerodynamic profiles follow established airfoil configurations or custom profiles developed through computational fluid dynamics optimization specifically for the low-wind-speed urban environments targeted by the system. The material and configuration choices enable reliable operation while minimizing maintenance requirements and maximizing system longevity under outdoor environmental exposure.

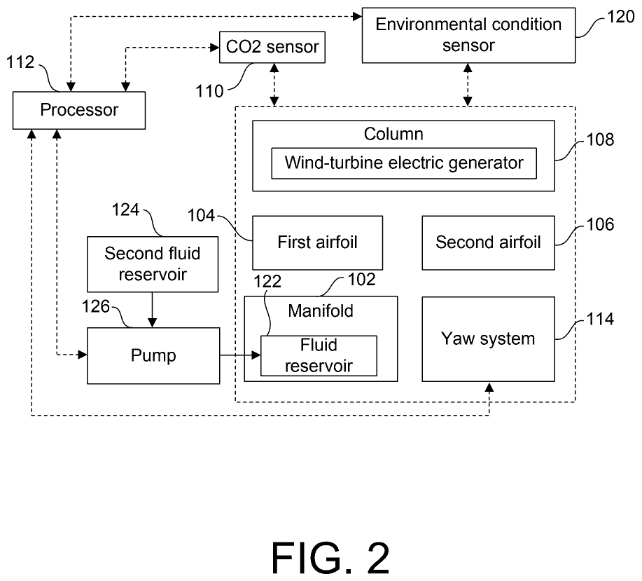

An exemplary block diagram of the system 100 is shown in FIG. 2. The block diagram of FIG. 2 particularly illustrates the interconnected nature of the system components and their respective data and control flows. The processor 112 serves as the central command unit, receiving input signals from both the CO₂ sensor 110 and the environmental condition sensor 120 via dedicated communication pathways. In some particular embodiments, these pathways comprise hardwired connections using industrial-grade cables rated for outdoor environmental conditions. In some embodiments, the communication occurs wirelessly that provide redundant communication paths. In some embodiments, the communication utilizes fiber optic connections for electromagnetic interference immunity. The processor 112 processes the incoming sensor data through embedded algorithms that implement the deep learning computations described herein. The processor 112 generates control signals that are transmitted to multiple system components simultaneously: the yaw system 114 for orientation control, the pump 126 for solution delivery control, and optionally to external monitoring systems (not shown) for data logging and remote oversight. The second fluid reservoir 124 maintains a predetermined volume of aqueous solution, typically ranging from 50 to 500 [IS THIS ACCURATE?] liters depending on the system size and expected CO₂ concentrations. In some embodiments, the reservoir capacity ranges from 100 to 200 liters for residential-scale installations. In some embodiments, the reservoir capacity exceeds 1000 liters for industrial-scale installations. The pump 126 includes flow rate control mechanisms that deliver precise volumes of solution to the manifold 102 based on real-time CO₂ readings, preventing both under-treatment of high-concentration air and waste of solution during low-concentration periods. The integrated control configuration enables autonomous operation with minimal human intervention while optimizing both energy production and CO₂ capture efficiency based on real-time environmental conditions.

The processing system comprises dedicated hardware components configured to implement the artificial intelligence algorithms described herein. In some embodiments, the processing system includes a central processing unit (CPU) with multiple cores operating at frequencies to handle the computational demands of real-time environmental data analysis. In some embodiments, the CPU comprises ARM-based processors optimized for low power consumption in outdoor installations. In some embodiments, the CPU comprises processors providing enhanced computational performance for complex deep learning algorithms.

The processing system further includes dedicated memory subsystems configured to store both program instructions and environmental data. In some embodiments, the memory comprises random access memory (RAM) to enable storage of neural network parameters and historical environmental measurements. In some embodiments, the memory includes error-correcting code (ECC) configurations to maintain data integrity in outdoor electromagnetic environments. In some embodiments, the memory comprises non-volatile storage devices such as solid-state drives (SSD) for long-term data storage and system operation logs.

In some embodiments, the processing system includes specialized hardware accelerators such as graphics processing units (GPU) or neural processing units (NPU) configured to accelerate the deep learning computations. The GPU configurations provide parallel processing capabilities with hundreds to thousands of processing cores optimized for matrix operations common in neural network implementations. In some embodiments, the NPU comprises dedicated silicon architectures specifically optimized for artificial intelligence workloads, providing computational efficiency improvements for neural network inference operations.

The processing system implements real-time operating system (RTOS) configurations to ensure deterministic response times for environmental control operations. In some embodiments, the RTOS provides task scheduling with microsecond-level precision to coordinate sensor data acquisition, neural network processing, and control signal generation. In some embodiments, the processing system includes timer circuits that monitor system operation and initiate automatic restarts in the event of software or hardware failures.

The processing system includes communication interfaces configured to enable data exchange with the environmental sensors and control systems. In some embodiments, the communication interfaces comprise serial interface protocols for sensor connectivity. In some embodiments, the interfaces include Ethernet connections providing network connectivity for remote monitoring and system updates. In some embodiments, the processing system includes cellular modem capabilities enabling connectivity in remote installation locations without existing network infrastructure.

Power management circuits within the processing system optimize energy consumption to minimize the load on the electrical generation system. In some embodiments, the power management includes dynamic voltage and frequency scaling that adjusts processor operating parameters based on computational workload requirements. In some embodiments, the power management includes sleep mode configurations that reduce power consumption during periods of minimal environmental activity while maintaining the ability to respond rapidly to changing conditions. The hardware configuration enables reliable operation in outdoor environments while maintaining the computational performance necessary for real-time artificial intelligence processing.

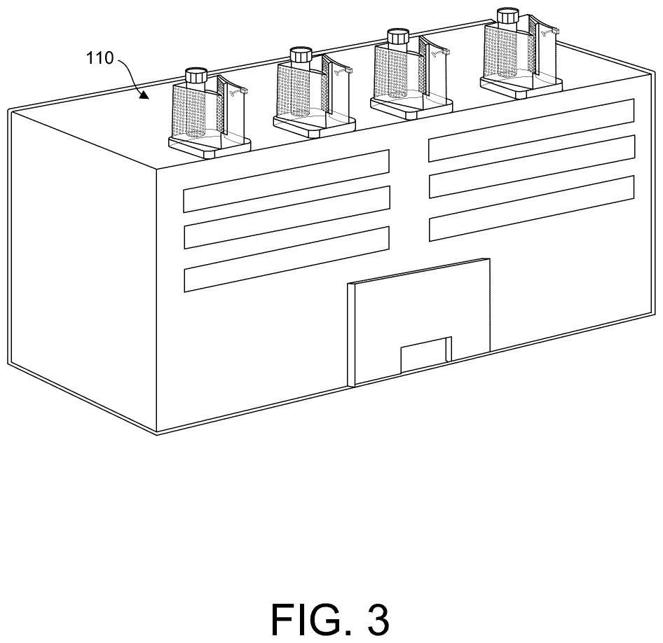

Because buildings facilitate the acceleration of wind as the wind ascends over the upper edges of the building, the aerodynamic structure of the edges enhances this wind acceleration which results in the formation of a low-pressure zone between the mirrored airfoil-pair 104, 106 and behind the column 108. This suction pulls air inward from the orifices 118 of the airfoils 104, 106 (forming air-jets), from the hollow airfoil interiors, supplied by the manifold 102, and also through the column 108 and past the wind-turbine propellor therein. The power is therefore generated by this subsequent internal flow stream. Once the polluted air has passed through the internal rotor, this air moves to the manifold 102 containing the aqueous solution (e.g., calcium hydroxide). As is known, once CO₂ mixes with calcium hydroxide, calcium carbonate and water result. The yaw system 114 is operated by the servomotor at the bottom of the structure (not shown). As will be described in greater detail below, the processor 112 is configured to continuously monitor and analyze wind direction data and to control the system 100 via the yaw system 114 accordingly. As shown in FIG. 3, a plurality of systems 100 may be positioned adjacent one another on top of the same structure to maximize the carbon capture and electric generation results.

The aerodynamic operation of the system 100 leverages the effect created by the specific geometric arrangement of the airfoils 104, 106 and column 108. As ambient air approaches the system 100, it encounters the leading edges of the airfoils 104, 106, which are shaped with optimized curvature profiles that promote smooth air flow attachment. The air accelerates as it passes between the airfoils 104, 106, creating a region of reduced pressure immediately behind the column 108. This pressure differential induces secondary air flows through the orifices 118 in the airfoils 104, 106, drawing air from the surrounding atmosphere into the hollow interior cavities. The pressure differential can be expressed mathematically as:

$$Δ P = ½ ρ ( {v}_{2}^{2} - {v}_{1}^{2} ) ,$$

where ΔP represents the pressure differential, p represents air density, v₁ represents the initial air velocity, and v₂ represents the accelerated air velocity between the airfoils. The pressure differential creates suction forces that draw additional air through the orifices 118, effectively increasing the total mass flow rate through the system beyond what would be achieved by ambient wind alone.

The inducted air combines with the primary air stream, increasing the total mass flow through the column 108 and past the propeller of the wind-turbine electric generation system. The total mass flow rate can be expressed as:

$$\dot{m} \_total + \dot{m} \_ambient + \dot{m} \_inducted ,$$

where m_total represents the total mass flow rate, m_ambient represents the ambient wind mass flow, and m_inducted represents the additional mass flow drawn through the orifices 118, 121. The enhanced air flow increases the rotational speed of the propeller, thereby increasing the electrical power output compared to conventional wind turbines of similar size.

The power generation relationship follows the equation:

$$P = ½ ρ {Av}^{3} Cp ,$$

where P represents the power output, ρ represents air density, A represents the swept area, v represents the air velocity, and Cp represents the power coefficient. The configuration enables higher effective air velocities (v) through the induced flow, resulting in cubic increases in power output.

Simultaneously, the air flow path directs the combined air stream through the manifold 102, where it contacts the aqueous calcium hydroxide solution. The chemical reaction occurs according to the equation:

$$Ca {( OH )}_{2} + {CO}_{2} \to {CaCO}_{3} + {H}_{2} O .$$

The reaction rate depends on several factors and can be expressed as:

$$r = k [ Ca {( OH )}_{2} ] [ {CO}_{2} ] ,$$

where r represents the reaction rate, k represents the rate constant, and the bracketed terms represent the concentrations of the reactants. The rate constant k varies with temperature according to the Arrhenius equation:

$$k = {Ae}^{⋀} ( - Ea / RT ) ,$$

where A represents the pre-exponential factor, Ea represents the activation energy, R represents the gas constant, and T represents the absolute temperature.

In some embodiments, the manifold 102 includes internal mixing elements that create turbulent flow patterns to enhance the gas-liquid contact efficiency. In some embodiments, the mixing elements comprise static mixers with helical configurations. In some embodiments, the mixing elements comprise perforated plates that create multiple contact zones. The mathematical framework enables precise prediction and optimization of both power generation and CO2 capture performance under varying environmental conditions.

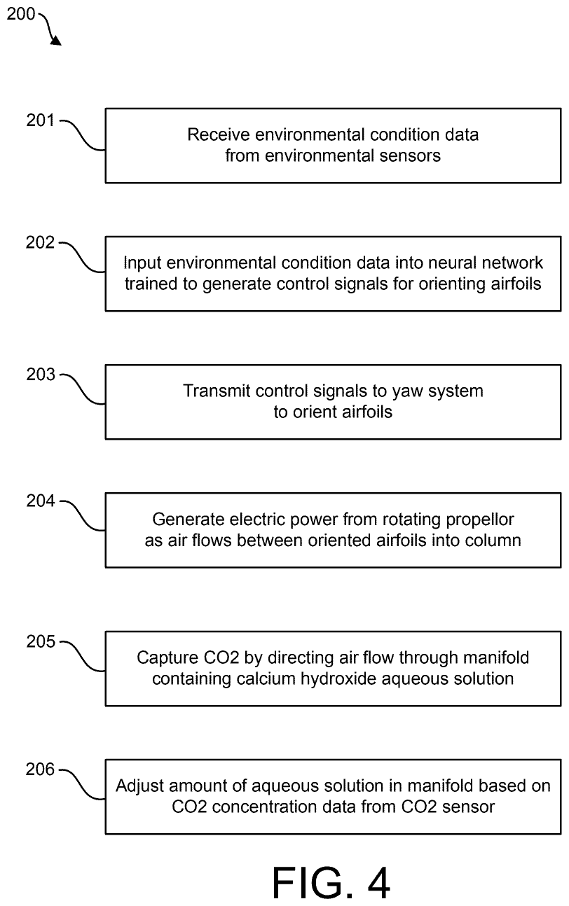

FIG. 4 illustrates a flow chart according to an example method 200 for smart electric power generation and CO₂ capturing, in accordance with the present disclosure. The method 200 may be performed by a system such as the system 100 described above with the reference to FIGS. 1 and 2. The method 200 proceeds through distinct operational phases, each with specific timing and performance criteria aligned with the deep learning and computational fluid dynamics (CFD) simulation capabilities described in the disclosure.

At operation 201, the processing system (e.g., the processor 112) receives environmental condition data from one or more environmental sensors 120. The environmental condition data collection occurs continuously at predetermined sampling intervals, typically ranging from 1 second to 60 seconds depending on the environmental variability and the requirements of the deep learning algorithms. In some embodiments, the sampling rate adapts dynamically based on the rate of change in environmental conditions, with higher sampling rates during rapidly changing conditions that require more frequent neural network processing. In some embodiments, the sampling rate increases to 10 Hz during wind gusts or storm conditions to capture transient environmental phenomena.

The environmental sensors 120 provide data with specified accuracy requirements that enable effective neural network training and inference operations. Wind speed measurements maintain accuracy within ±0.1 m/s, wind direction measurements maintain accuracy within ±2 degrees, and CO₂ concentration measurements maintain accuracy within ±10 ppm. The sensor 120 accuracy enables precise control decisions according to measurement uncertainty propagation calculations that account for the cumulative effects of individual sensor uncertainties on the overall system performance predictions.

At operation 202, the environmental condition data is input into a neural network trained to generate control signals for orienting the pair of air foil blades 104, 106 based upon the environmental condition data. The neural network processing implements the deep learning algorithms described in the disclosure, utilizing the augmented data sets comprising CFD simulation results and real-time sensor measurements. The neural network input preprocessing includes data normalization to standard ranges compatible with the training data distributions, outlier detection and filtering to remove erroneous measurements that could compromise prediction accuracy, and temporal windowing to capture recent trends in environmental conditions relevant to the pattern recognition algorithms. In some embodiments, the outlier detection utilizes statistical methods with thresholds set at multiple standard deviations from established measurement ranges. In some embodiments, the temporal windowing captures data over rolling time periods of 5 to 30 minutes to provide sufficient context for the deep learning pattern recognition.

The neural network processing generates control predictions based on the learned relationships between environmental conditions and optimal system performance. The prediction algorithms implement mathematical transformations that map the current environmental state to recommended control actions, utilizing the knowledge base developed through CFD simulation training data. In some embodiments, the predictions include confidence metrics that indicate the reliability of the recommended actions based on the similarity between current conditions and the training data scenarios.

At operation 203, the processing system transmits the generated control signals to the yaw system 114 to selectively rotate the pair of airfoils 104, 106 so as to orient the same. The control signal transmission to the yaw system 114 includes safety interlocks and validation algorithms that ensure safe system operation. The safety interlocks implement logic conditions that prevent system operation during extreme environmental conditions that could damage equipment or compromise safety. In some embodiments, the maximum wind speed thresholds are based on the structural ratings of the system components. In some embodiments, the maximum rotation rates are limited to 5 degrees per second to ensure smooth orientation changes that maintain optimal air flow patterns.

At operation 204, electric power is generated from the rotating propellor of the wind-turbine generator as the air flows between the oriented air foil blades 104, 106 into the column 108 housing the propellor and the rotor. The electric power generation monitoring provides real-time feedback that validates the effectiveness of the AI-driven control decisions. The power monitoring implements feedback loops that compare actual power generation with the predictions from the neural network algorithms, enabling continuous validation and potential adjustment of the control strategies. The monitoring data contributes to the ongoing learning process by providing performance feedback that can be used to refine the neural network parameters during operation.

At operation 205, CO₂ is captured from the air flowing through the system 100 by directing the air flow through the manifold 102 containing the aqueous solution (e.g., calcium hydroxide). The CO₂ capture process operates under control algorithms that optimize solution delivery based on the environmental measurements and CO₂ concentration predictions from the deep learning system. The solution delivery follows the chemical reaction stoichiometry of the Ca(OH)₂+CO₂→CaCO₃+H₂O process, with delivery rates calculated to maintain optimal reaction conditions based on the predicted CO₂ loading from the environmental analysis.

At operation 206, an amount of the aqueous solution delivered to the manifold 102 can be adjusted based on CO₂ concentration data from the CO₂ sensor 110. The solution delivery adjustment implements feedback control based on moving average calculations and pattern recognition from the AI algorithms. The adjustment process utilizes both short-term measurements and longer-term pattern predictions to optimize chemical consumption while maintaining treatment effectiveness. In some embodiments, the adjustment algorithms implement predictive control that anticipates CO₂ concentration changes based on environmental patterns learned through the deep learning process. The comprehensive method configuration enables autonomous system operation with performance optimization through artificial intelligence while maintaining equipment safety and longevity.

During operation of the wind-turbine based electric generation, the processing system (e.g., the processor 112) utilizing one or more artificial intelligence (AI) algorithms can be configured to dynamically predict the wind direction at the location of the systems 100 to allow the systems 100 to adjust their orientations in real time to optimize their alignment with the changing wind direction, thereby maximizing energy capture efficiency. Once the polluted air reaches the manifold 102 with the aqueous solution, the calcium hydroxide reacts with CO₂ in the presence of water to form calcium carbonate (CaCO₃), a solid precipitate. CO₂ from the post combustion processes reacts with calcium hydroxide in the solution to form calcium carbonate. This reaction effectively captures and sequesters CO₂ from the polluted air, preventing its release into the atmosphere. The process is low-cost due to its low energy consumption since a minimum amount of energy is needed to power the sensors 120 needed for the AI and the injection of the solution into the manifold 102, which is selectively controlled by the processor 112 based on received data from the sensor 120.

Real-time data from the high-frequency sensors 120, such as wind direction, wind speed, relative humidity, atmospheric turbulence, and CO₂ concentration, may be integrated into the deep learning algorithms for the output generation by the processor 112. These localized environmental measurements from the sensors 120 augment the input data for AI-based prediction models, enhancing their accuracy and responsiveness.

It is worth noting that additional operations or variations may be included in the method 200, depending on the specific requirements of the system. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

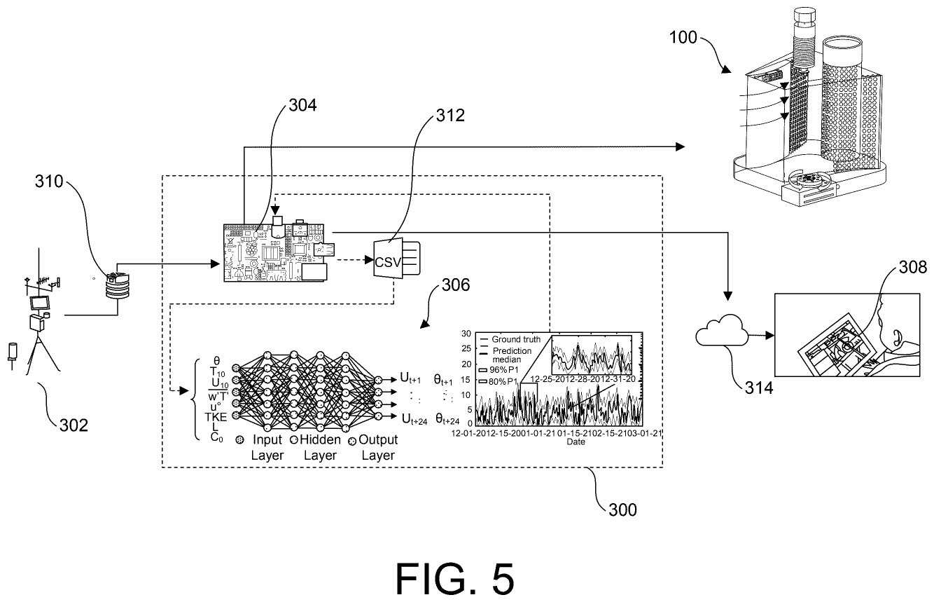

FIG. 5 illustrates an exemplary AI-based control system 300 configured for dynamic environmental responsiveness in the operation of the carbon capture and power generation system 100. As shown in FIG. 5, deep neural networks are trained on augmented data sets comprising computational fluid dynamics (CFD) simulation results and sensor measurements to rapidly predict wind flow patterns and CO₂ dispersion dynamics. These predictions are approximated to real-time conditions and incorporated into the wind turbine control system through optimization methodologies to dynamically adjust turbine positioning and operation parameters. The system 300 utilizes real-time predictions to optimize turbine positioning and operation in response to rapid changes in environmental conditions. By maximizing power output and CO₂ filtration efficiency, the system contributes to both energy production and environmental sustainability objectives.

The AI-based control system 300 integrates a weather station 302 with environmental sensors 120, a microcontroller 304, a deep neural network model 306, and interfaces 308 for downstream control and monitoring systems. As depicted in FIG. 5, a weather station 302 provides real-time environmental data, including wind speed, direction, and other atmospheric conditions, via an industrial protocol 310 (e.g., MODBUS). This data is received by a microcontroller 304, which serves as the local edge computing unit. The microcontroller 304 executes scripts (e.g., written in Python) to preprocess the incoming data and store it in local memory 312 (e.g., as CSV files). These datasets are then input into a trained deep neural network model 306 embedded within the AI-based control architecture. The neural network model 306 comprises input, hidden, and output layers configured to analyze historical and real-time environmental variables (e.g., temperature θ, wind speed U, turbulence intensity T_I, turbulent kinetic energy (TKE), Obukhov length L, and CO₂ concentration C_0). The model predicts future states of the environment, such as wind speed and direction at multiple future time intervals (e.g., U_{t+1}, θ_{t+1}, U_{t+24}, θ_{t+24}), enabling proactive system control. The AI-generated predictions are then used to determine optimal control actions for adjusting the positioning of the air foil blades 104, 106 via the yaw system 114. Control signals are issued through industrial communication protocols such as OPC (Open Platform Communications) to a servomotor that implements PID (Proportional-Integral-Derivative) control logic to orient the system 100. Additionally, the control architecture supports cloud 314 integration for remote end-user monitoring, allowing system performance visualization and control override. The integration of predictive AI-based control ensures that the carbon capture and energy generation subsystems dynamically adjust to varying wind conditions with minimal latency. This results in maximized power output and CO₂ filtration efficiency under fluctuating environmental conditions.

The deep learning algorithms referenced in the disclosure comprise neural network architectures specifically configured for environmental time-series data processing. The neural networks implement pattern recognition algorithms that analyze temporal sequences of environmental measurements to predict optimal system positioning and operation parameters. In some embodiments, the neural networks comprise feedforward architectures with multiple hidden layers configured to process static environmental measurements. In some embodiments, the neural networks comprise recurrent architectures configured to analyze temporal sequences and identify patterns in environmental data over time periods ranging from minutes to hours.

The neural network architectures process input data vectors comprising normalized environmental measurements. In some embodiments, the neural networks implement convolutional layers configured to identify spatial patterns in environmental data when multiple sensors are distributed around the installation site.

In some embodiments, the neural networks implement recurrent layers configured to maintain memory states that capture temporal dependencies in environmental measurements. The recurrent processing enables the system to learn patterns such as daily wind direction cycles, seasonal variations in atmospheric conditions, and short-term weather trend predictions. The recurrent computations process sequential data according to formulations that update internal memory states based on both current inputs and previous memory contents.

The neural network training process utilizes the CFD simulation results described in the disclosure as training data to teach the system optimal responses to various environmental conditions. In some embodiments, the training data comprises millions of simulation scenarios covering wind speeds from 0 to 30 m/s, wind directions across 360 degrees, and atmospheric conditions including temperature ranges from −40° C. to +60° C. and humidity levels from 0% to 100%. In some embodiments, the training process implements supervised learning algorithms such as backpropagation with gradient descent optimization to adjust network parameters based on the difference between predicted and optimal system responses.

In some embodiments, the neural networks implement attention mechanisms that focus computational resources on the most relevant environmental measurements for the current decision-making process. The attention mechanisms weight different input measurements according to their relevance, enabling the system to prioritize wind direction sensors during rapidly changing conditions while focusing on CO₂ concentration measurements during high-pollution events.

The neural network inference operations execute within computational time budgets of 10 to 100 milliseconds to enable real-time system response to changing environmental conditions. In some embodiments, the inference time scales with network complexity, with simpler feedforward networks processing inputs in under 10 milliseconds while more complex recurrent networks require up to 100 milliseconds for comprehensive temporal pattern analysis. The computational efficiency enables continuous operation without significant energy consumption overhead. The AI implementation enables the system 100 to adapt to local environmental patterns and optimize performance based on site-specific conditions learned through continuous operation.

In some embodiments, a capture efficiency exceeds 90% when the AI algorithms optimize solution flow rates for specific environmental conditions and CO₂ concentration patterns. In some embodiments, the system maintains capture efficiency above 80% even during peak pollution events with CO₂ concentrations exceeding 1000 ppm through predictive chemical delivery control. The efficiency demonstrates minimal degradation over operating periods exceeding 6 months between major maintenance intervals.

The AI-driven orientation system may demonstrates orientation error reductions compared to conventional wind tracking systems, particularly during turbulent wind conditions common in urban environments where buildings create complex air flow patterns.

In some embodiments, the response time decreases to under 10 seconds when the AI algorithms detect rapid environmental changes requiring immediate system response. In some embodiments, the predictive capabilities of the deep learning algorithms enable anticipatory positioning based on environmental trend analysis, further improving system responsiveness. In some embodiments, the system includes remote monitoring through network connectivity that enables predictive maintenance scheduling and performance optimization.

The calcium carbonate precipitate management follows the stoichiometric predictions from the chemical reaction equation, with removal intervals ranging from 30 to 90 days depending on CO₂ capture rates and local environmental conditions. The precipitate accumulation rate enables predictive maintenance scheduling based on the chemical reaction monitoring and AI-predicted environmental loading. In some embodiments, the precipitate removal includes automated flushing systems that operate during low-wind periods identified by the AI algorithms. The performance characteristics validate the practical viability of the dual-function system for commercial deployment while achieving measurable environmental benefits through both renewable energy generation and direct atmospheric CO₂ removal in urban environments.

Accordingly, the system 100 and the method in accordance with the present disclosure therefore provide for smart CO₂ capture which (1) utilizes AI for a maximized performance of the apparatus since the higher the CO₂ concentration, the higher the efficiency of the apparatus; (2) increases energy production efficiency through proactive turbine positioning and operation optimization; (3) increases CO₂ filtration rates by dynamically adapting to changing environmental conditions; (4) improves responsiveness to localized weather patterns and urban topography for optimized turbine placement; and (5) reduces CO₂ emissions and environmental impact, contributing to sustainability goals.

Reference systems that may be used herein can refer generally to various directions (for example, upper, lower, forward and rearward), which are merely offered to assist the reader in understanding the various embodiments of the disclosure and are not to be interpreted as limiting. Other reference systems may be used to describe various embodiments, such as those where directions are referenced to the portions of the device, for example, toward or away from a particular element, or in relations to the structure generally (for example, inwardly or outwardly).

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used in combination with some or all of the features of other embodiments as would be understood by one of ordinary skill in the art, whether or not explicitly described as such. One or more exemplary embodiments have been shown and described, and all changes and modifications that come within the spirit of the disclosure are desired to be protected.

I/We claim:

1. A system for electric power generation and capturing carbon dioxide (CO₂), comprising:
a pair of air foil blades spaced apart to allow environmental air flow between them, wherein each air foil blade of the pair of air foil blades includes a hollow interior cavity and a plurality of orifices on an exterior surface of the air foil blade, wherein each orifice of the plurality of orifices is configured to allow air flow from an exterior position relative to the air foil blade through the orifices and into the hollow interior cavity;
an electric generation system, wherein the electric generation system includes at least a rotor and a propellor coupled with the rotor, wherein the electric generation system is configured to generate and output an electric power signal as the propellor rotates;
a manifold having a first fluid reservoir for containing an aqueous solution to capture CO₂ from the air flowing through the system;
a yaw system selectively operable to rotate the air foil blades;
one or more environmental sensors configured to monitor one or more environmental conditions and generate respective output data signals;
a processing system having access to a neural network, wherein the neural network is trained to receive the output data signals from the one or more environmental sensors and generate rotational control signals to the yaw system to rotate the air foil blades based upon the output data signals, wherein the processing system is configured to selectively transmit the rotational control signals to the yaw system.

2. The system of claim 1, wherein the electric generation system is housed within a column including a plurality of orifices on an exterior surface of the column.

3. The system of claim 2, wherein the air foil blades and the column are positioned to create a low-pressure zone that accelerates airflow through the plurality of orifices of the air foil blades and the column.

4. The system of claim 3, wherein the column is positioned between the air foil blades.

5. The system of claim 1, wherein the processing system is configured to perform real time computational fluid dynamics (CFD) calculations using the output data signals from the one or more environmental sensors and generate CFD results, wherein the processing system is configured to input the CFD results into the neural network.

6. The system of claim 1, wherein the processing system is configured to receive wind direction data and adjust orientation of the air foil blades to align with prevailing wind patterns.

7. The system of claim 1, wherein the aqueous solution includes calcium hydroxide.

8. The system of claim 2, wherein the exterior surface of each air foil blade and/or the exterior surface of the column is/are at least partially coated with titanium dioxide (TiO₂) to disintegrate nitrogen oxides (NOx) in the surrounding air.

9. The system of claim 1, further comprising a CO₂ sensor, wherein the CO₂ sensor is configured to monitor environmental CO₂ conditions and generate an output CO₂ data signal.

10. The system of claim 9, further comprising:
a second fluid reservoir storing the aqueous solution; and
a pump system for transferring the aqueous solution from the second fluid reservoir to the first fluid reservoir, wherein the pump system is configured to transfer portions of the aqueous solution from the second fluid reservoir to the first fluid reservoir based upon the output CO₂ data signal to control the aqueous solution delivery to the manifold.

11. The system of claim 1, wherein the yaw system comprises a servomotor configured to rotate the air foil blades about a vertical axis.

12. The system of claim 1, wherein the one or more environmental sensors include a sonic anemometer configured to monitor wind speed and direction.

13. The system of claim 10, wherein the processing system is communicably coupled to the one or more environmental sensors and/or the CO₂ sensor via a wireless connection.

14. The system of claim 13, wherein the pump system is configured to be operated by the processing system upon the processing system analyzing the output CO₂ data signal.

15. The system of claim 5, wherein the neural network is trained using augmented data comprising both the output data signals of the one or more environmental sensors and the CFD results.

16. A system for electric power generation and capturing carbon dioxide (CO₂), comprising:
a pair of air foil blades spaced apart to allow environmental air flow between them, each air foil blade of the pair of air foil blades including a hollow interior cavity and orifices on an exterior surface of the air foil blade, each of the orifices configured to allow air flow from an exterior position relative to the air foil blade into the hollow interior cavity;
an electric generation system housed within a column including orifices on an exterior surface of the column, wherein the electric generation system includes a rotor and a propellor coupled with the rotor, and is configured to generate and output an electric power signal as the propellor rotates when the air flows between the air foil blades into the column;
a manifold for containing a calcium hydroxide aqueous solution to capture CO₂ from the air flowing through the system;
a yaw system selectively operable to rotate the air foil blades;
one or more environmental sensors configured to monitor one or more environmental conditions and generate respective output data signals;
a processing system having access to a neural network, wherein the neural network is trained to receive the output data signals from the one or more environmental sensors and generate rotational control signals to the yaw system to rotate the air foil blades based upon the output data signals, wherein the processing system is configured to selectively transmit the rotational control signals to the yaw system.

17. A method for electric power generation and capturing carbon dioxide (CO₂), the method comprising:
receiving, by a processing system, environmental condition data from one or more environmental sensors;
inputting, by the processing system, the environmental condition data into a neural network trained to generate control signals for orienting a pair of air foil blades based upon the environmental condition data, the pair of air foil blades being spaced apart, configured to allow environmental air flow between them;
transmitting, by the processing system, the control signals to a yaw system to selectively rotate the pair of air foil blades so as to orient the air foil blades;
generating electric power from a propellor coupled to a rotor when the propellor rotates as the air flows between the oriented air foil blades into a column housing the propellor and the rotor; and
capturing CO₂ from the air flowing through a system of the air foil blades and the column, by directing the air flow through a manifold containing an aqueous solution adapted to capture CO₂.

18. The method of claim 17, wherein the aqueous solution includes calcium hydroxide.

19. The method of claim 17, further comprising adjusting an amount of the aqueous solution delivered to the manifold based on CO₂ concentration data from a CO₂ sensor configured to monitor environmental CO₂ conditions.

20. The method of claim 17, further comprising performing, by the processing system, real time computational fluid dynamics (CFD) analysis based on the environmental condition data and using results of the CFD analysis as input to the neural network.
